(19) United States
(12) Patent Application Publication
(10) Pub. No.: US 2025/0177653 A1
(43) Pub. Date: Jun. 5, 2025

(54) CARTRIDGE HOLDER ADAPTED FOR A CARTRIDGE BODY AND A PLUNGER OF A CARTRIDGE ASSEMBLY AND RELATED INJECTION SYSTEM

(71) Applicant: ALTEK BIOTECHNLOGY CORPORATION, Hsinchu City (TW)

(72) Inventors: Yu-Cheng Huang, Hsinchu City (TW); Yi Chu, Hsinchu City (TW)

(21) Appl. No.: 18/527,361

(22) Filed: Dec. 3, 2023

(51) Int. Cl. A61M 5/28

(52) U.S. Cl. CPC A61M 5/28

(57) ABSTRACT

A cartridge holder adapted for a cartridge body and a plunger of a cartridge assembly is provided and includes a holding component, a first cooperating component, a second cooperating component and a resilient component. The holding component is configured to accommodate the cartridge assembly. The first cooperating component is mounted on the holding component and immovable relative to the holding component. The second cooperating component is movable relative to the first cooperating component and configured to be driven to rotate to disengage from the first cooperating component. The resilient component is disposed between the first cooperating component and the second cooperating component and configured to drive the second cooperating component to push the plunger to slide relative to the cartridge body along a first direction after the second cooperating component is driven to rotate to disengage from the first cooperating component. Besides, a related injection system is also provided.

### BACKGROUND OF THE INVENTION

### 1. Field of the Invention

The present invention relates to a cartridge product and a related injection system, and more specifically, to a cartridge holder adapted for a cartridge body and a plunger of a cartridge assembly, and a related injection system.

### 2. Description of the Prior Art

An injection system, e.g., a pen injector or a wearable injector, is a medical device designed for drug delivery. In order to ensure a proper operation of the drug delivery, the injection system is usually equipped with a forcing mechanism for enabling a plunger to overcome resistance, i.e., a break-loose force, between the plunger and a cartridge, so as to dislodge the plunger relative to the cartridge from a resting position. However, the conventional injection systems having such forcing mechanisms, that are available in the market, fail to meet requirements of simple structure and easy operation. Therefore, an improvement of the injection system is urgently needed.

### SUMMARY OF THE INVENTION

It is an objective of the present invention to provide a cartridge holder adapted for a cartridge body and a plunger of a cartridge assembly, and a related injection system for solving the aforementioned problem.

In order to achieve the aforementioned objective, the present invention discloses a cartridge holder adapted for a cartridge body and a plunger of a cartridge assembly. The cartridge holder includes a holding component, a first cooperating component, a second cooperating component and a resilient component. The holding component is configured to accommodate the cartridge assembly. The first cooperating component is mounted on the holding component and immovable relative to the holding component. The second cooperating component is movable relative to the first cooperating component and configured to be driven to rotate to disengage from the first cooperating component. The resilient component is disposed between the first cooperating component and the second cooperating component and configured to drive the second cooperating component to push the plunger to slide relative to the cartridge body along a first direction after the second cooperating component is driven to rotate to disengage from the first cooperating component.

According to an embodiment of the present invention, the holding component includes a first end and a second end opposite to the first end. An inserting portion is formed on the first end for an insertion of an inserting component, and the first cooperating component is located adjacent to the second end.

According to an embodiment of the present invention, the second cooperating component is at least partially movably disposed between the first cooperating component and the first end of the holding component.

According to an embodiment of the present invention, the resilient component is at least partially accommodated inside one of the first cooperating component and the second cooperating component.

According to an embodiment of the present invention, the second cooperating component includes a cooperating structure, and the cooperating structure includes an inclined surface configured to be abutted along a second direction opposite to the first direction for driving the second cooperating component to rotate.

According to an embodiment of the present invention, the cooperating structure is at least partially exposed out of the holding component.

According to an embodiment of the present invention, the holding component includes a slot structure, and the cooperating structure is at least partially exposed out of the holding component through the slot structure.

According to an embodiment of the present invention, the slot structure extends along a longitudinal direction of the holding component. The slot structure includes a guiding portion and an exposing portion connected to the guiding portion, and the cooperating structure is at least partially exposed out of the holding component through the exposing portion.

According to an embodiment of the present invention, the first cooperating component includes a first engaging structure. The second cooperating component includes a second engaging structure configured to engage with the first engaging structure, and the second engaging structure is further configured to be driven to disengage from the first engaging structure by a rotating movement of the second cooperating component.

According to an embodiment of the present invention, the first cooperating component includes a first locking structure. The holding component includes a second locking structure configured to engage with the first locking structure for restraining a movement of the first cooperating component.

In order to achieve the aforementioned objective, the present invention further discloses an injection system. The injection system includes an injector body and a cartridge device detachably mounted on the injector body. The injector body includes an abutting component. The cartridge device includes a cartridge assembly and a cartridge holder for holding the cartridge assembly. The cartridge assembly includes a cartridge body and a plunger slidable relative to the cartridge body. The cartridge holder includes a holding component, a first cooperating component, a second cooperating component and a resilient component. The holding component is configured to accommodate the cartridge assembly. The first cooperating component is mounted on the holding component and immovable relative to the holding component. The second cooperating component is movable relative to the first cooperating component and configured to be driven to rotate to disengage from the first cooperating component by the abutting component when the cartridge device slides relative to the injector body along a first direction. The resilient component is disposed between the first cooperating component and the second cooperating component and configured to drive the second cooperating component to push the plunger to slide relative to the cartridge body along the first direction after the second cooperating component is driven to rotate to disengage from the first cooperating component by the abutting component.

In summary, the present invention utilizes the abutting component to rotate the second cooperating component to disengage from the first cooperating component and further utilizes the resilient component to drive the second cooperating component to push the plunger to slide relative to the cartridge body along the first direction, by overcoming the resistance, i.e., the break-loose force, between the plunger and the cartridge body after the second cooperating component is driven to rotate to disengage from the first cooperating component. Therefore, the present invention has advantages of simple structure and easy operation.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram of an injection system as a cartridge device is detached from an injector body according to an embodiment of the present invention.

FIG. 2 is an exploded diagram of the cartridge device according to the embodiment of the present invention.

FIG. 3 is a diagram of a holding component according to the embodiment of the present invention.

FIG. 4 is a diagram of a first cooperating component, a second cooperating component and a resilient component according to the embodiment of the present invention.

FIG. 5 is a diagram of the injection system in a first used state according to the embodiment of the present invention.

FIG. 6 is a partial diagram of the cartridge device as the injection system is in the first used state according to the embodiment of the present invention.

FIG. 7 is a diagram of the injection system in a second used state according to the embodiment of the present invention.

FIG. 8 is a partial diagram of the cartridge device as the injection system is in the second used state according to the embodiment of the present invention.

FIG. 9 is a diagram of the injection system in a third used state according to the embodiment of the present invention.

FIG. 10 is a partial diagram of the cartridge device as the injection system is in the third used state according to the embodiment of the present invention.

### DETAILED DESCRIPTION

In the following detailed description of the preferred embodiments, reference is made to the accompanying drawings which form a part hereof, and in which is shown by way of illustration specific embodiments in which the invention may be practiced. In this regard, directional terminology, such as “top”, “bottom”, “left”, “right”, “front”, “back”, etc., is used with reference to the orientation of the Figure(s) being described. The components of the present invention can be positioned in a number of different orientations. As such, the directional terminology is used for purposes of illustration and is in no way limiting. Accordingly, the drawings and descriptions will be regarded as illustrative in nature and not as restrictive. Also, if not specified, the term “connect” or “coupled” is intended to mean either an indirect or direct electrical/mechanical connection. Thus, if a first device is connected or coupled to a second device, that connection may be through a direct electrical/mechanical connection, or through an indirect electrical/mechanical connection via other devices and connections.

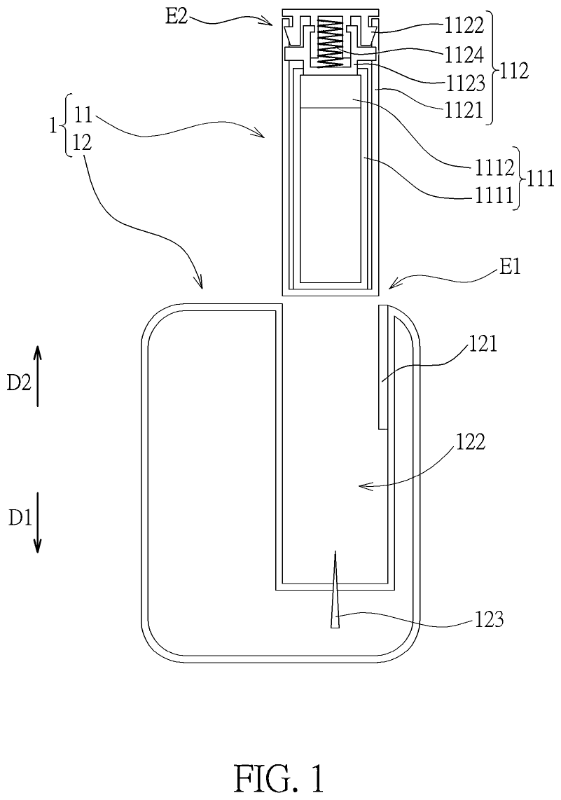

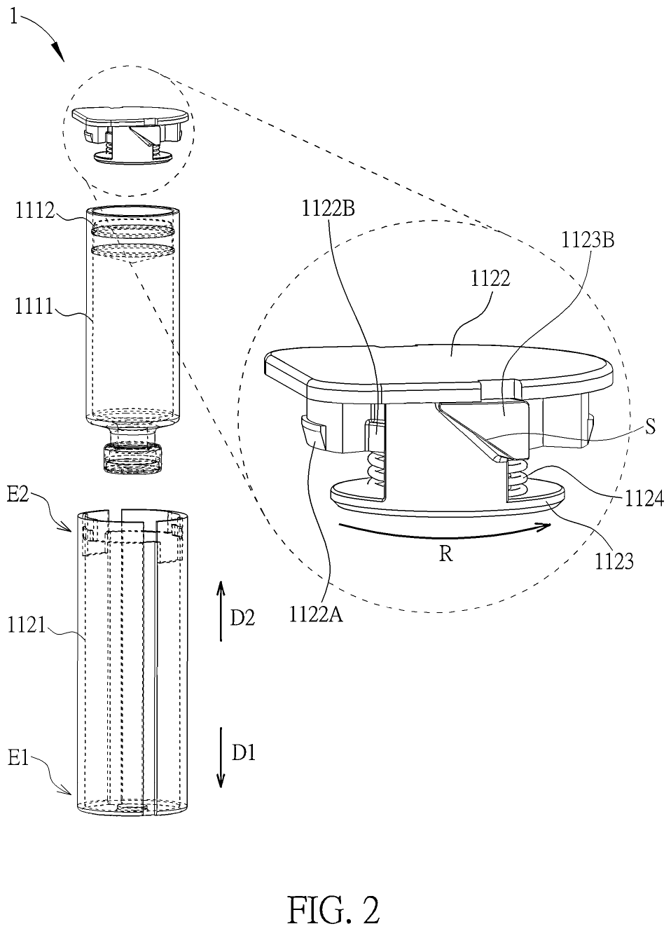

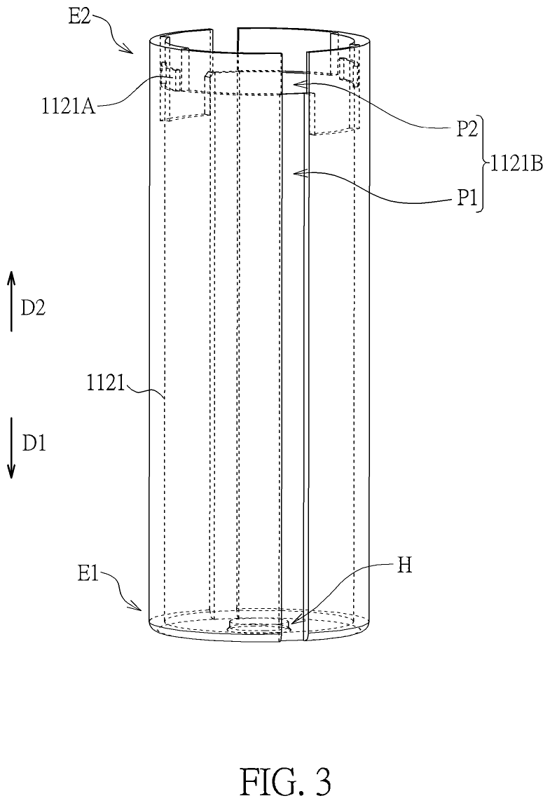

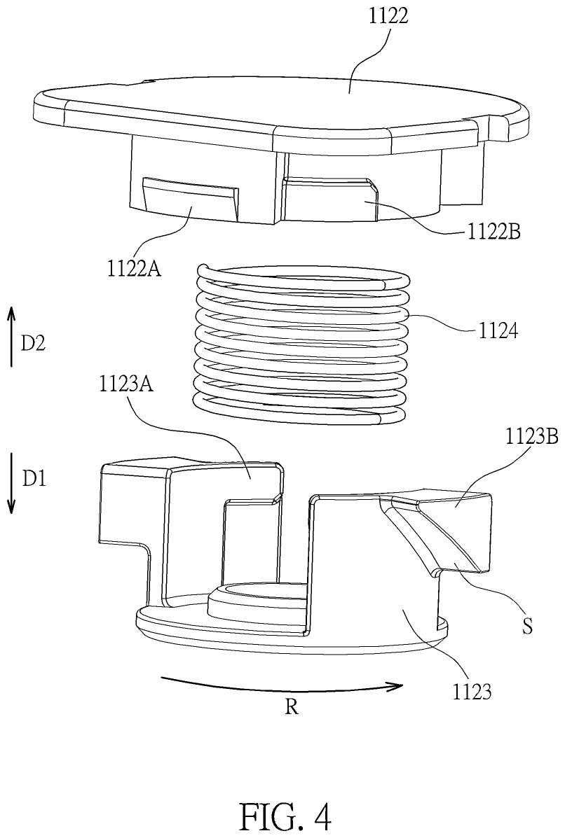

Please refer to FIG. 1 to FIG. 4. FIG. 1 is a diagram of an injection system 1 as a cartridge device 11 is detached from an injector body 12 according to an embodiment of the present invention. FIG. 2 is an exploded diagram of the cartridge device 11 according to the embodiment of the present invention. FIG. 3 is a diagram of a holding component 1121 according to the embodiment of the present invention. FIG. 4 is a diagram of a first cooperating component 1122, a second cooperating component 1123 and a resilient component 1124 according to the embodiment of the present invention. As shown in FIG. 1 to FIG. 4, the injection system 1 includes the cartridge device 11 and the injector body 12. The cartridge device 11 is detachably mounted on the injector body 12, e.g., in a sliding manner. The injector body 12 includes an abutting component 121, an accommodating structure 122 and an inserting component 123. The accommodating structure 122 is for at least partially accommodating the cartridge device 11. The abutting component 121 is disposed on a lateral wall of the accommodating structure 122 and for cooperating with the cartridge device 11. The inserting component 123 is disposed on a bottom wall of the accommodating structure 122 and can be inserted into the cartridge device 11 for allowing drug to flow out of the cartridge device 11 through the inserting component 123. In this embodiment, the abutting component 121 can be a bump structure formed in a rod shape, and the inserting component 123 can be a hollow needle. However, the present invention is not limited to this embodiment.

Furthermore, as shown in FIG. 1 to FIG. 4, the cartridge device 11 includes a cartridge assembly 111 and a cartridge holder 112 for holding the cartridge assembly 111. The cartridge assembly 111 includes a cartridge body 1111 and a plunger 1112 slidable relative to the cartridge body 1111. The cartridge holder 112 includes the holding component 1121, the first cooperating component 1122, the second cooperating component 1123 and the resilient component 1124. The holding component 1121 is configured to provide an accommodating space for accommodating the cartridge assembly 111. The first cooperating component 1122 is mounted on the holding component 1121 and immovable relative to the holding component 1121. The second cooperating component 1123 is releasably engaged with the first cooperating component 1122 and movable relative to the first cooperating component 1122. The resilient component 1124 is disposed between the first cooperating component 1122 and the second cooperating component 1123 for biasing the second cooperating component 1123 to push the plunger 1112 to slide relative to the cartridge body 1111. As shown in FIG. 1 to FIG. 4, the second cooperating component 1123 is configured to be driven to rotate along a rotating direction R to disengage from the first cooperating component 1122 by the abutting component 121 when the cartridge device 11 slides relative to the injector body 12 along a first direction D1, and the second cooperating component 1123 is further configured to be driven by the resilient component 1124 to push the plunger 1112 to slide relative to the cartridge body 1111 along the first direction D1 by overcoming resistance, i.e., a break-loose force, between the plunger 1112 and the cartridge body 1111 after the second cooperating component 1123 is driven to rotate along the rotating direction R to disengage from the first cooperating component 1122 by the abutting component 121.

In this embodiment, as shown in FIG. 1 to FIG. 4, the holding component 1121 includes a first end E1 and a second end E2 opposite to the first end E1. An inserting portion H is formed on the first end E1 for insertion of the inserting component 123 into the cartridge body 1111, and the first cooperating component 1122 is located adjacent to the second end E2. Specifically, the inserting portion H includes a through hole structure, and the inserting component 123 can be inserted into the cartridge body 1111 by passing through the through hole structure of the inserting portion H when the cartridge device 11 is slidably mounted on the injector body 12.

As shown in FIG. 2 to FIG. 4, in order to position the first cooperating component 1122 on the holding component 1211, the first cooperating component 1122 includes a first locking structure 1122A, and the holding component 1121 includes a second locking structure 1121A formed on the second end E2 of the holding component 1121. The second locking structure 1121A is configured to engage with the first locking structure 1122A for restraining a movement of the first cooperating component 1122. In this embodiment, by way of example, the first locking structure 1122A can be a locking lug, and the second locking structure 1121A can be a locking recess. However, the present invention is not limited to this embodiment.

Furthermore, the second cooperating component 1123 is at least partially movably disposed between the first cooperating component 1122 and the first end E1 of the holding component 1121. The resilient component 1124 is at least partially accommodated inside one of the first cooperating component 1122 and the second cooperating component 1123 for biasing the second cooperating component 1123 to push the plunger 1112. In this embodiment, by way of example, the second cooperating component 1123 is at least partially movably disposed between the first cooperating component 1122 and the plunger 1112, and two ends portions of the resilient component 1124 are accommodated inside the first cooperating component 1122 and the second cooperating component 1123, respectively. However, the present invention is not limited to this embodiment.

As shown in FIG. 2 and FIG. 4, the first cooperating component 1122 further includes a first engaging structure 1122B. The second cooperating component 1123 includes a second engaging structure 1123A. The second engaging structure 1123A can be engaged with the first engaging structure 1122B for restraining the second cooperating component 1123 from being driven by the resilient component 1124 along the first direction D1. When the second cooperating component 1123 is driven by the abutting component 121 to rotate along the rotating direction R, the second engaging structure 1123A can be disengaged from the first engaging structure 1122B for allowing the second cooperating component 1123 to be driven by the resilient component 1124 along the first direction D1. In this embodiment, by way of example, the first engaging structure 1122B can be a step-shaped recess, and the second engaging structure 1123A can be a step-shaped protrusion. However, the present invention is not limited to this embodiment.

Besides, as shown in FIG. 2 to FIG. 4, in order to achieve the second cooperating component 1123 to be driven by the abutting component 121 to rotate along the rotating direction R, the second cooperating component 1123 includes a cooperating structure 1123B, and the cooperating structure 1123B includes an inclined surface S configured to be abutted by the abutting component 121 along a second direction D2 opposite to the first direction D1 for driving the second cooperating component 1123 to rotate along the rotating direction R when the cartridge device 11 is mounted on the injector body 12. Specifically, the cooperating structure 1123B is at least partially exposed out of the holding component 1121, such that the abutting component 121 can abut against the inclined surface S of the cooperating structure 1123B to drive the second cooperating component 1123 to rotate along the rotating direction R. In this embodiment, by way of example, the holding component 1121 includes a slot structure 1121B, and the cooperating structure 1123B is at least partially exposed out of the holding component 1121 through the slot structure 1121B. Specifically, the slot structure 1121B extends along a longitudinal direction of the holding component 1121 parallel to the first direction D1. The slot structure 1121B includes a guiding portion P1 and an exposing portion P2 connected to the guiding portion P1 and located adjacent to the second end E2 of the holding component 1121, and the cooperating structure 1123B is at least partially exposed out of the holding component 1121 through the exposing portion P2.

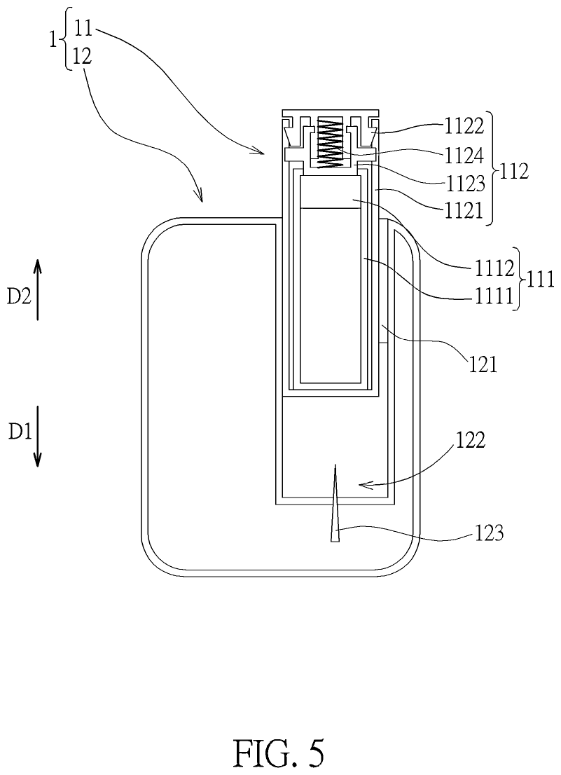

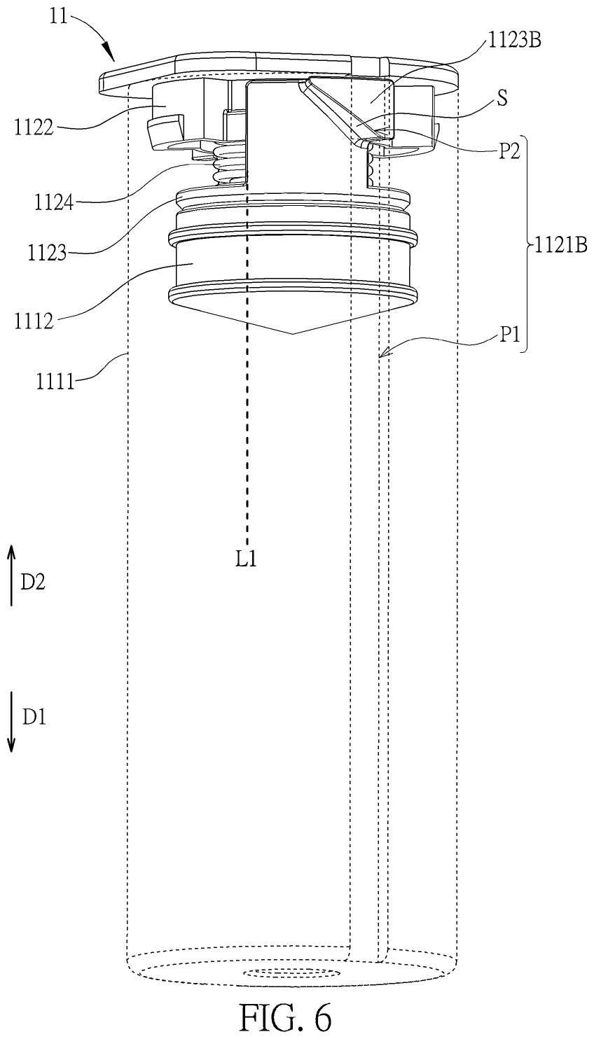

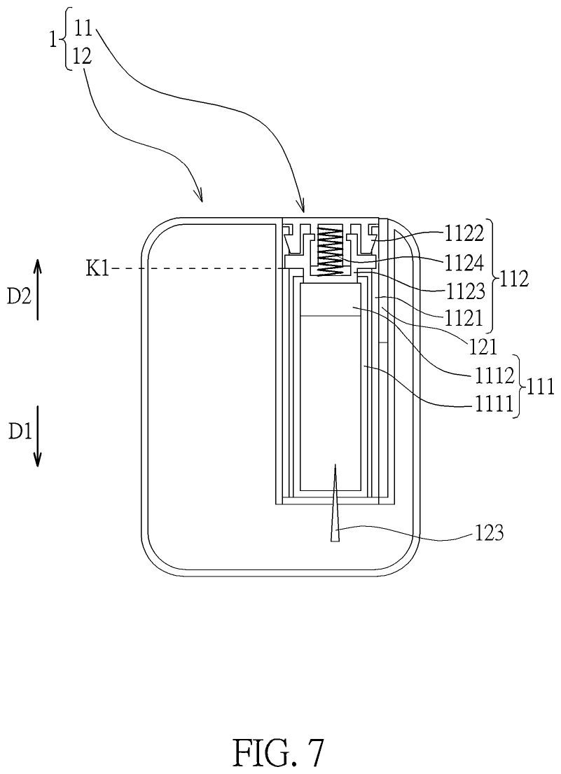

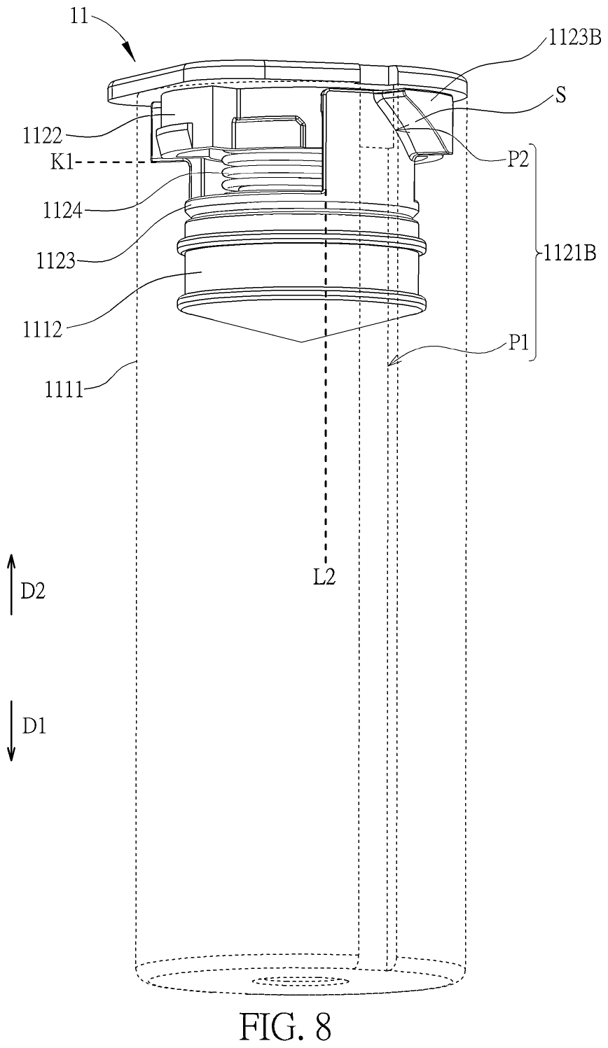

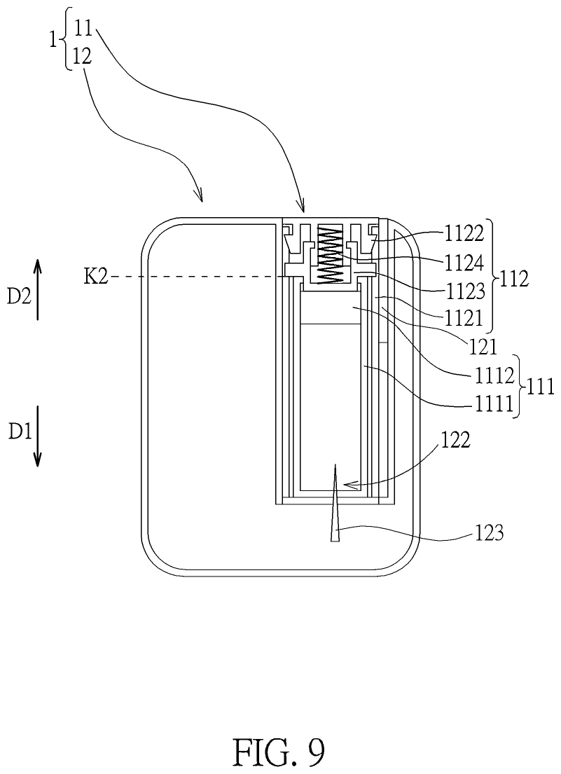

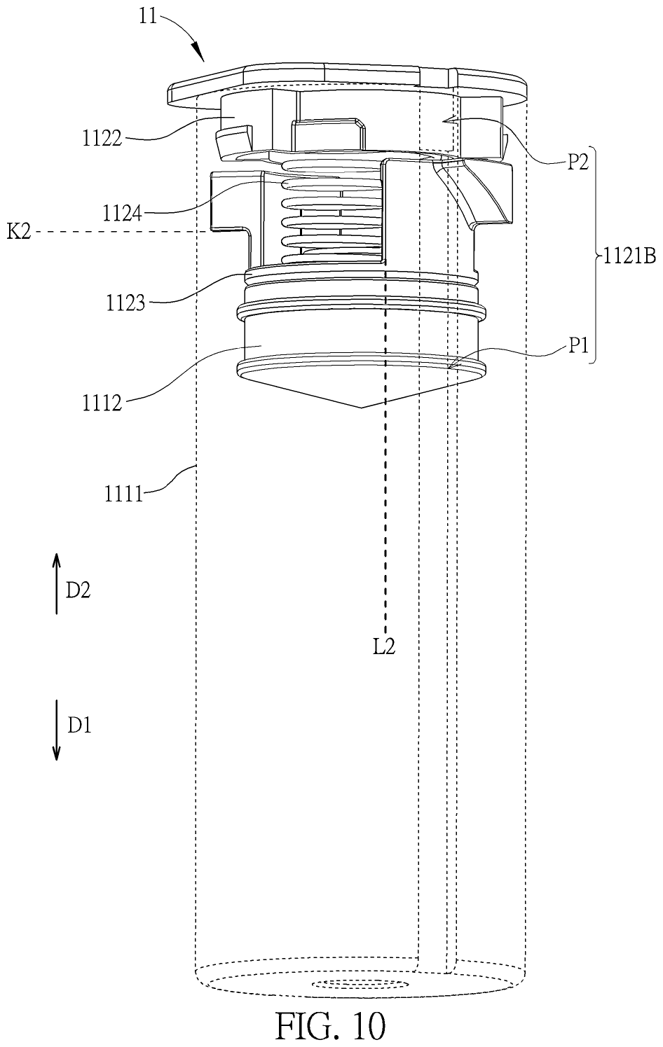

Please refer to FIG. 5 to FIG. 10. FIG. 5 is a diagram of the injection system 1 in a first used state according to the embodiment of the present invention. FIG. 6 is a partial diagram of the cartridge device 11 as the injection system 1 is in the first used state according to the embodiment of the present invention. FIG. 7 is a diagram of the injection system 1 in a second used state according to the embodiment of the present invention. FIG. 8 is a partial diagram of the cartridge device 11 as the injection system 1 is in the second used state according to the embodiment of the present invention. FIG. 9 is a diagram of the injection system 1 in a third used state according to the embodiment of the present invention. FIG. 10 is a partial diagram of the cartridge device 11 as the injection system 1 is in the third used state according to the embodiment of the present invention. Detailed description of the present invention is provided as follows. When the cartridge device 11 is mounted on the injector body 12, the abutting component 121 slides along the slot structure 1121B from the guiding portion P1 of the slot structure 1121B to the exposing portion P2 of the slot structure 1121B, such that the inclined surface S of the cooperating structure 1123B is abutted by the abutting component 121 to drive the second cooperating component 1123 to rotate from an engaging position as shown in FIG. 5 and FIG. 6 to a disengaging position as shown in FIG. 7 and FIG. 8 along the rotating direction R for disengaging the second cooperating component 1123 from the first cooperating component 1122. After the second cooperating component 1123 is rotated to the disengaging position as shown in FIG. 7 and FIG. 8, the second cooperating component 1123 can be driven by the resilient component 1124 to move from an initial position K1 as shown in FIG. 7 and FIG. 8 to a predetermined position K2 as shown in FIG. 9 and FIG. 10 along the first direction D1, so as to push the plunger 1112 to slide relative to the cartridge body 1111 along the first direction D1, by overcoming the resistance i.e., the break-loose force, between the plunger 1112 and the cartridge body 1111, which can prevent the plunger 1112 from being stuck, so as to ensure an proper operation of the drug delivery.

In contrast to the prior art, the present invention utilizes the abutting component to rotate the second cooperating component to disengage from the first cooperating component and further utilizes the resilient component to drive the second cooperating component to push the plunger to slide relative to the cartridge body along the first direction, by overcoming the resistance, i.e., the break-loose force, between the plunger and the cartridge body after the second cooperating component is driven to rotate to disengage from the first cooperating component. Therefore, the present invention has advantages of simple structure and easy operation.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A cartridge holder adapted for a cartridge body and a plunger of a cartridge assembly, the cartridge holder comprising:
a holding component configured to accommodate the cartridge assembly;
a first cooperating component mounted on the holding component and immovable relative to the holding component;
a second cooperating component movable relative to the first cooperating component and configured to be driven to rotate to disengage from the first cooperating component; and
a resilient component disposed between the first cooperating component and the second cooperating component and configured to drive the second cooperating component to push the plunger to slide relative to the cartridge body along a first direction after the second cooperating component is driven to rotate to disengage from the first cooperating component.

2. The cartridge holder of claim 1, wherein the holding component comprises a first end and a second end opposite to the first end, an inserting portion is formed on the first end for an insertion of an inserting component, and the first cooperating component is located adjacent to the second end.

3. The cartridge assembly of claim 2, wherein the second cooperating component is at least partially movably disposed between the first cooperating component and the first end of the holding component.

4. The cartridge holder of claim 1, wherein the resilient component is at least partially accommodated inside one of the first cooperating component and the second cooperating component.

5. The cartridge holder of claim 1, wherein the second cooperating component comprises a cooperating structure, and the cooperating structure comprises an inclined surface configured to be abutted along a second direction opposite to the first direction for driving the second cooperating component to rotate.

6. The cartridge holder of claim 5, wherein the cooperating structure is at least partially exposed out of the holding component.

7. The cartridge holder of claim 6, wherein the holding component comprises a slot structure, and the cooperating structure is at least partially exposed out of the holding component through the slot structure.

8. The cartridge holder of claim 7, wherein the slot structure extends along a longitudinal direction of the holding component, the slot structure comprises a guiding portion and an exposing portion connected to the guiding portion, and the cooperating structure is at least partially exposed out of the holding component through the exposing portion.

9. The cartridge holder of claim 1, wherein the first cooperating component comprises a first engaging structure, the second cooperating component comprises a second engaging structure configured to engage with the first engaging structure, and the second engaging structure is further configured to be driven to disengage from the first engaging structure by a rotating movement of the second cooperating component.

10. The cartridge holder of claim 1, wherein the first cooperating component comprises a first locking structure, the holding component comprises a second locking structure configured to engage with the first locking structure for restraining a movement of the first cooperating component.

11. An injection system comprising:
an injector body comprising an abutting component; and
a cartridge device detachably mounted on the injector body, the cartridge device comprising:
a cartridge assembly comprising a cartridge body and a plunger slidable relative to the cartridge body; and
a cartridge holder for holding the cartridge assembly, the cartridge holder comprising:
a holding component configured to accommodate the cartridge assembly;
a first cooperating component mounted on the holding component and immovable relative to the holding component;
a second cooperating component movable relative to the first cooperating component and configured to be driven to rotate to disengage from the first cooperating component by the abutting component when the cartridge device slides relative to the injector body along a first direction; and
a resilient component disposed between the first cooperating component and the second cooperating component and configured to drive the second cooperating component to push the plunger to slide relative to the cartridge body along the first direction after the second cooperating component is driven to rotate to disengage from the first cooperating component by the abutting component.

12. The injection system of claim 11, wherein the holding component comprises a first end and a second end opposite to the first end, an inserting portion is formed on the first end for an insertion of an inserting component, and the first cooperating component is located adjacent to the second end.

13. The injection system of claim 12, wherein the second cooperating component is at least partially movably disposed between the first cooperating component and the first end of the holding component.

14. The injection system of claim 11, wherein the resilient component is at least partially accommodated inside one of the first cooperating component and the second cooperating component.

15. The injection system of claim 11, wherein the second cooperating component comprises a cooperating structure, and the cooperating structure comprises an inclined surface configured to be abutted by the abutting component along a second direction opposite to the first direction for driving the second cooperating component to rotate.

16. The injection system of claim 15, wherein the cooperating structure is at least partially exposed out of the holding component.

17. The injection system of claim 16, wherein the holding component comprises a slot structure, and the cooperating structure is at least partially exposed out of the holding component through the slot structure.

18. The injection system of claim 17, wherein the slot structure extends along a longitudinal direction of the holding component, the slot structure comprises a guiding portion and an exposing portion connected to the guiding portion, and the cooperating structure is at least partially exposed out of the holding component through the exposing portion.

19. The injection system of claim 11, wherein the first cooperating component comprises a first engaging structure, the second cooperating component comprises a second engaging structure configured to engage with the first engaging structure, and the second engaging structure is further configured to be driven to disengage from the first engaging structure by a rotating movement of the second cooperating component.

20. The injection system of claim 11, wherein the first cooperating component comprises a first locking structure, the holding component comprises a second locking structure configured to engage with the first locking structure for restraining a movement of the first cooperating component.
